(19) United States
(12) Patent Application Publication
(10) Pub. No.: US 2007/0032851 A1
(43) Pub. Date: Feb. 8, 2007

(54) Protection by electroactive polymer sleeve

(75) Inventors: James Lee Shippy III, Plymouth, MN (US); Tracee Eidenschink, Wayzata, MN (US); Karl A. Jagger, Deephaven, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: Boston Scientific Scimed, Inc., Maple Grove, MN (US)

(21) Appl. No.: 11/195,381

(22) Filed: Aug. 2, 2005

(51) Int. Cl. A61F 2/06

(52) U.S. Cl. 623/1.12

(57) ABSTRACT

A system for protecting a stent includes an electroactive polymer (EAP) sleeve, a stent, a balloon catheter, and a voltage source. A voltage is applied to the EAP sleeve, whereupon the EAP sleeve expands. The stent, disposed about the balloon catheter is inserted into the region defined by the inner surface of the EAP sleeve. The voltage is removed, whereupon the EAP sleeve contracts.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Stents are often deployed to a location within a body lumen or vessel through the use of a stent delivery system. Such systems often comprise an elongate catheter about which the stent is mounted prior to deployment of the stent. A stent delivery system is assembled prior to use by crimping the stent onto a region of the catheter.

Existing crimping devices and methods are described in, for example, U.S. Pat. No. 6,387,118; U.S. Pat. No. 6,108,886; U.S. Pat. No. 6,092,273; U.S. Pat. No. 6,082,990; U.S. Pat. No. 6,074,381; U.S. Pat. No. 6,063,102; U.S. Pat. No. 5,992,000; etc.

An electroactive polymer refers to a polymer that acts as an insulating dielectric between two electrodes and may deflect upon application of a voltage difference between the two electrodes. Electroactive polymers (EAP) are materials such as polypyrrole, polyalanine, polyacetylene, polythiophene and polyvinylidene difluoride (PVDF), etc. that show shape deformation when an electric field is applied. Electroactive polymer materials can be manufactured such that when there is a voltage difference between the two electrodes, the EAP material increases in volumetric size. Alternatively, the EAP material can be manufactured such that when there is a voltage difference between the two electrodes, the material decreases in volumetric size. When an electrical field is applied across the EAP, the EAP deforms as a result of stresses generated by the movement of water and mobile positive ions in the polymer.

Existing electroactive polymers are described in U.S. Pat. No. 6,515,077, U.S. Pat. No. 6,545,391, and U.S. Pat. No. 6,664,718, for example.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All U.S. patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In some embodiments, the present invention is concerned with the crimping and otherwise reducing in size of stents, including drug delivery or coated stents of any configuration or expansion type, including inflation expandable stents, self-expanding stents, hybrid expandable stents, etc. For the purpose of this disclosure, it is understood that the term ‘stent’ includes stents, stent-grafts, grafts and vena cava filters and other implantable medical devices for luminal support. It is also understood that the term ‘crimping’ refers to a reduction in size or profile of a stent and/or a device upon which it is to be mounted; and ‘crimper’ refers to devices for accomplishing such reduction in size or profile of same.

In at least one embodiment the invention is directed towards a variety of embodiments, including a system for protecting a stent comprising a stent, a balloon catheter, and an electroactive polymer (EAP) sleeve or crimper. A voltage source, electrically connected to the EAP material and a conductive member via contacts, applies a voltage across the contacts. The EAP material is thereby activated and expands. The stent, disposed about the balloon catheter, is inserted into the expanded sleeve. Once completely inserted, the voltage source is removed, causing the EAP material to contract. A radial compression force is exerted on the stent assembly as a result of the contraction.

In some embodiments the EAP sleeve may be comprised of multiple layers. For example, in a multi-layer embodiment there may be more than one layer of an EAP, more than one layer of material that is not an EAP, or more than one layer of both EAP and non-EAP material. When a voltage is applied to such an embodiment, the multiple layers could cause the combination to bend or twist, rather than expand. The multi-layered combination could take on a variety of shapes and forms, depending on the desired arrangement of the layers.

In at least one embodiment, the conductive member is a sleeve upon which the EAP material is attached. In another embodiment, the conductive member can be wires distributed throughout the EAP material. In some embodiments the conductive member can be ribbons distributed throughout the EAP material. Or, in other embodiments wires or ribbons may be wrapped around the exterior of the EAP material.

The EAP material can be formed in a substantially cylindrical form in one embodiment. In this embodiment, the EAP material is substantially evenly distributed such that a cross-section of the material is ring shaped. In other embodiments, the EAP material may be formed in patterns that are not ring shaped, such as trapezoidal. These trapezoidal patterns may help reduce stresses that are not directed radially inward. That is, by allowing sufficient spacing between each section of EAP material formed in a trapezoidal pattern, the EAP material may expand and apply substantially radial forces while minimizing the forces applied to nearby trapezoidal sections.

Some embodiments of the present invention include a lubricious coating. After the stent and balloon catheter are inserted into the EAP sleeve and the voltage is applied, the EAP sleeve exerts a radial force on the stent as the EAP sleeve constricts. A lubricious coating can be applied to the inner surface of the EAP sleeve prior to insertion of the stent and balloon catheter, thereby reducing the risk of damaging the stent coating during EAP constriction.

In some embodiments of the present invention, the stent is crimped prior to insertion into the EAP sleeve. If the stent is not crimped prior to insertion into the EAP sleeve, an embodiment of the present invention accomplishes crimping, provided that the EAP sleeve can deliver sufficient compressive forces. In some embodiments, EAP activation can take place at substantially the same time as stent crimping by a mechanical tool, thereby significantly reducing or eliminating, “bunching” fold creases that are prevalent in current methods. In other embodiments, the stent is not crimped prior to insertion into the EAP sleeve.

Another embodiment envisions packaging and shipping the assembly with the EAP sleeve attached. That is, at time the stent assembly is ready to use, the medical personnel could open the packaging and then turn the voltage source to an on state, expanding the EAP sleeve. The medical personnel can remove the assembly from the EAP sleeve, thereby reducing the risk of damaging the stent coating prior to use.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE DRAWINGS

A detailed description of the invention is hereafter described with specific reference being made to the drawings.

FIG. 1 is a side view of an electroactive polymer sleeve in an expanded state.

FIG. 2 is a side view of an electroactive polymer sleeve in an unexpanded state.

FIG. 3 is a side view of a stent.

FIG. 4 is a side view of a balloon catheter.

FIG. 5 is a side view of the stent of FIG. 3 disposed about the balloon catheter of FIG. 4.

FIG. 6 is a side view of the electroactive polymer sleeve of FIG. 1 disposed about the stent and balloon catheter combination of FIG. 5.

FIG. 6a is a cross-sectional view of the embodiment of FIG. 6.

FIG. 7 is a side view of the electroactive polymer sleeve of FIG. 2 disposed about the stent and balloon catheter combination of FIG. 5.

FIG. 7a is a cross-sectional view of the embodiment of FIG. 7.

FIG. 8 is a cross-section of one embodiment of an electroactive polymer sleeve with insulated wires distributed therein.

FIG. 9 is a cross-section of one embodiment of the electroactive polymer sleeve.

FIG. 10 is a cross-section of one embodiment of an electroactive polymer sleeve with insulated ribbons distributed therein.

FIG. 11 is a side view of the electroactive polymer with an insulated wire disposed about the electroactive polymer.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, and/or substituted for, elements depicted in another figure as desired

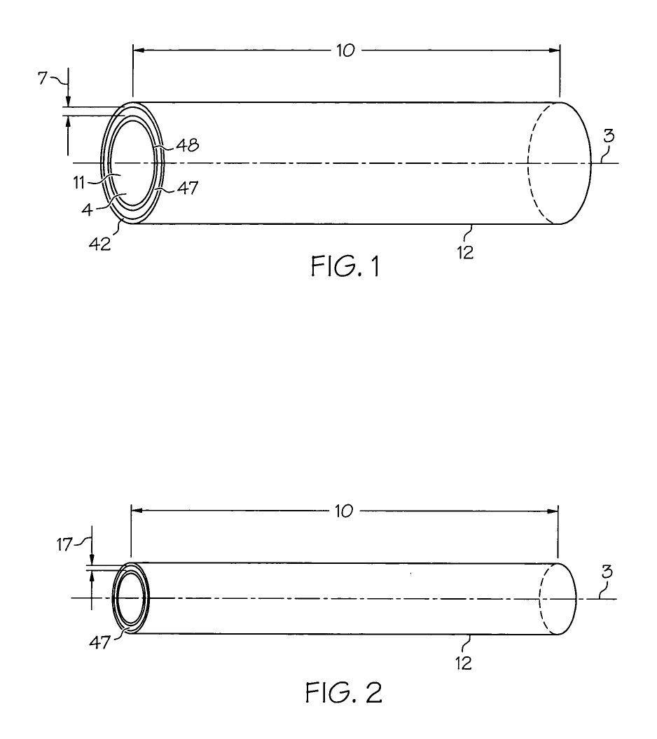

FIG. 1 depicts an embodiment of an EAP sleeve, shown generally at 12. The EAP sleeve 12 includes an EAP material 47 disposed between first conductive member 42 and second conductive member 48. The EAP sleeve 12 of FIG. 1 is in an expanded state and has a length 10 relative to axis 3. The EAP material of FIG. 1 also has a thickness 7 and may include a lubricious coating 11 on the inner surface 4. A lubricious coating can be applied to the inner surface 4 of the EAP sleeve prior to insertion of a stent and balloon catheter, thereby reducing the risk of damaging the stent coating during EAP constriction.

FIG. 2 depicts the EAP sleeve 12 shown in FIG. 1, but in an unexpanded state. In its unexpanded state, the EAP sleeve 12 has a length 10 relative to axis 3, length 10 being substantially the same as in unexpanded state length 10 shown in FIG. 1. Furthermore, the EAP material 47 shown in FIG. 2 has a thickness 17 which is less than expanded state thickness 7.

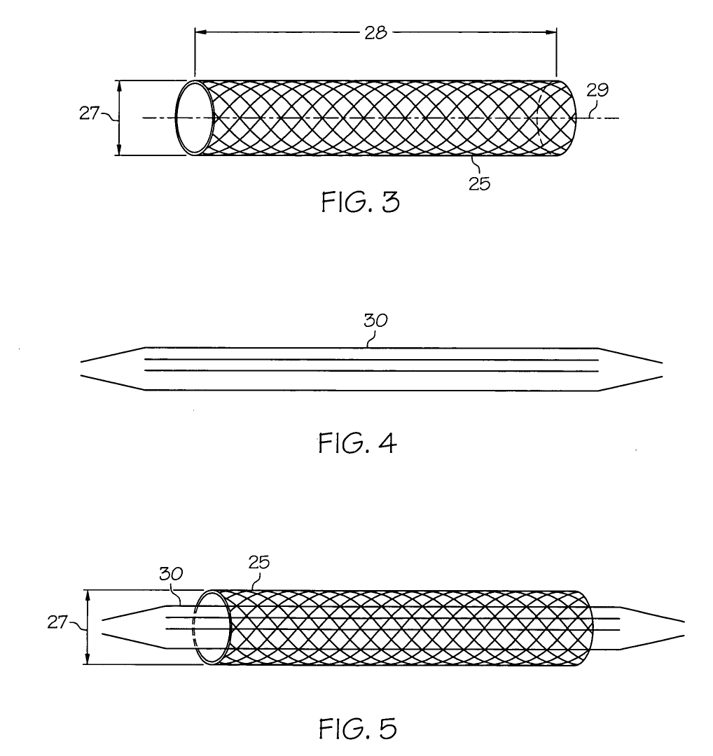

A stent 25 is shown in FIG. 3. Stent 25 has a length 28 relative to axis 29. Also, stent 25 has an outer diameter 27.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

In FIG. 5 the stent 25 of FIG. 3 is shown disposed about the balloon catheter 30 of FIG. 4.

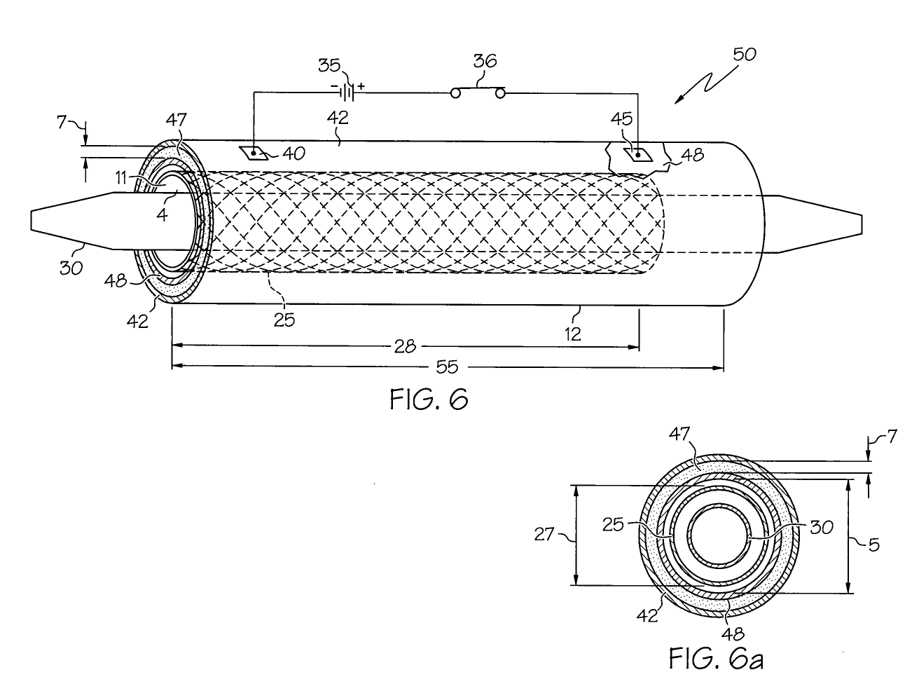

FIG. 6 depicts an embodiment of the present system for protecting a stent assembly, shown generally at 50, in an expanded state. The expanded EAP sleeve of FIG. 1 is shown generally at 12. The EAP sleeve 12 may also include a lubricious coating 11 distributed on inner surface 4. The EAP sleeve 12 is expanded by applying a voltage, supplied by a voltage source 35, across first conductive member 42 and second conductive member 48. This may be accomplished by attaching contact 40 to conductive member 42 and contact 45 to conductive member 48, as shown. Alternatively, the voltage source 35 may connected directly to conductive members 42 and 48. Conductive member 42 and conductive member 48 are separated by EAP material 47. Closed switch 36 and contacts 40 and 45 are shown only to indicate conceptually how voltage can be applied to conductive members; a number of more practical methods of applying a voltage across the EAP material may be available. In this embodiment, conductive member 42 is a first conductive sheath shown disposed about the EAP material 47; EAP material 47 is disposed about a second conductive sheath, conductive member 48. Many other embodiments of conductive members are possible, such as wires, ribbons, or both, dispersed within the EAP material. Also, conductive members such wires, ribbons, or both can be disposed about the EAP material.

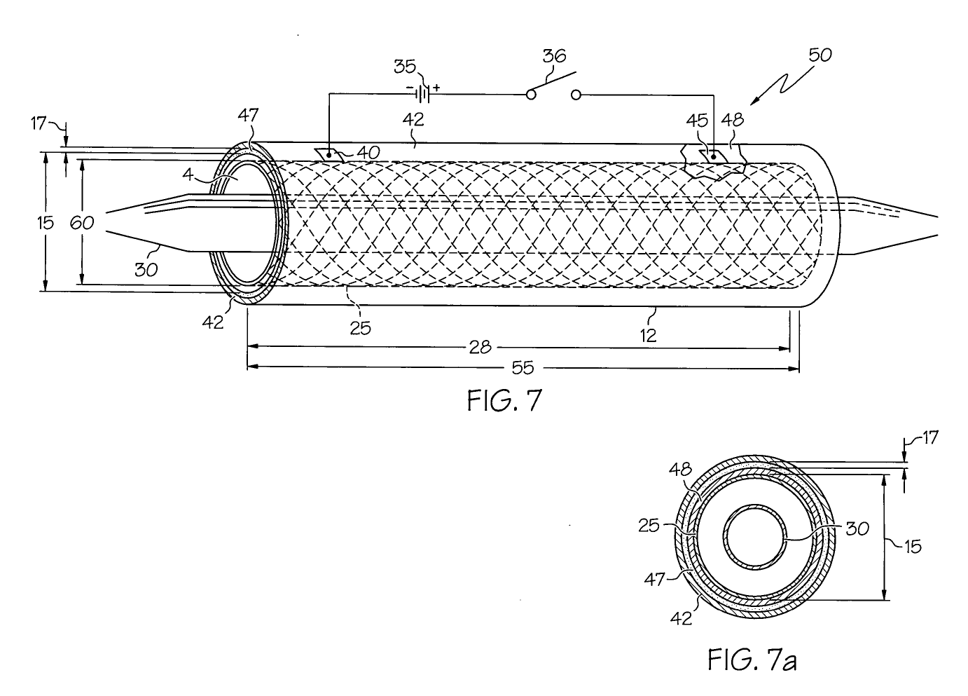

After voltage source 35 is applied, the thickness 17 of the EAP material 47 in its unexpanded state, shown in FIGS. 7 and 7a, increases to thickness 7, shown in FIGS. 6 and 6a. Conductive members 42 and 48 can be ductile and change shape with EAP material 47. Also, the inner diameter 15 of the EAP sleeve in its unexpanded state, shown in FIG. 7, increases to inner diameter 5, as shown in FIG. 6a. In the expanded state, as shown in FIG. 6, axial length 55 of EAP sleeve 12 is no less than axial length 28 of stent 25, thereby providing a protective covering for the stent.

FIG. 7 depicts an embodiment of the present system for protecting a stent assembly, shown generally at 50, in an unexpanded state. The unexpanded EAP sleeve of FIG. 1 is shown generally at 12. As depicted by open switch 36, voltage source 35 is not applied across contacts 40 and 45 when EAP sleeve 12 is in the unexpanded state. In its unexpanded state, the EAP material has thickness 17. Unexpanded state thickness 17 is less than the expanded state thickness 5 that is shown in FIGS. 6 and 6a. Contact 40 is connected to a first conductive member 42 and contact 45 is connected to a second conductive member 48, separated by EAP material 47. Open switch 36 and contacts 40 and 45 are shown only to indicate conceptually how a voltage can be easily removed from an EAP material. In the unexpanded state, EAP sleeve 12 has an inner diameter 15 defined by inner surface 4. A substantially radial compression force is exerted from EAP sleeve 12 to stent 25, causing outer diameter 27 of stent 25 of FIG. 6a to reduce to outer diameter 60, as shown in FIG. 7. However, axial length 55 of EAP sleeve 12 is no less than axial length 28 of stent 25, thereby providing a protective covering for the stent.

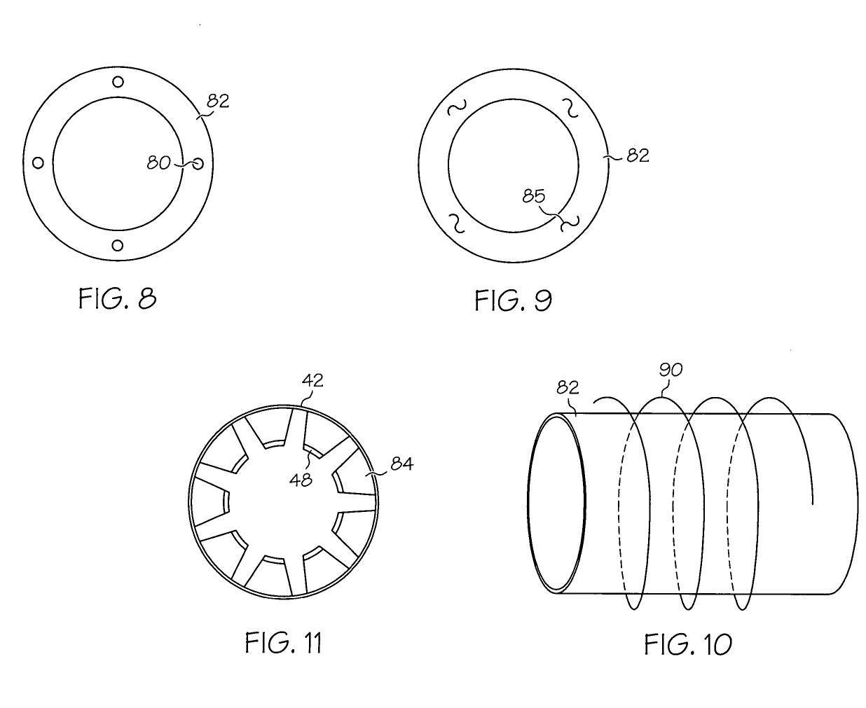

As shown in the cross-section of FIG. 8, some embodiments may use wires 80 embedded in EAP material 82 in order to expand the EAP material. Other embodiments instead may use ribbons 85 embedded in the EAP material 82, as shown in FIG. 9. In some embodiments, the wire/ribbon 90 may not be embedded in EAP material 82, but is instead wrapped around the exterior of EAP material 82, as in FIG. 10.

Regarding the EAP material, in some embodiments it is formed such that a cross-section is substantially ring shaped, depicted by reference numeral 82 in FIG. 8. However, a number of other patterns are possible, such as a pattern that includes a number of trapezoids 84 when the material is cross-sectioned, as shown in FIG. 11. In FIG. 11, a EAP material 84 is disposed between first conductive member 42 and second conductive member 48. A trapezoidal pattern could reduce the amount of stress produced that is not directed radially inward. That is, by allowing sufficient spacing between each trapezoidal section 84 of EAP material, the EAP material may expand and apply substantially radial forces while minimizing the forces applied to nearby trapezoidal sections. The trapezoidal pattern shown in FIG. 11 is meant only to exemplify one possible pattern. Numerous other patterns are possible, such as semi-circular or rectangular.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A system for protecting an assembly comprising:
a stent;
a balloon catheter, the balloon catheter comprising a catheter shaft and a balloon, the balloon being folded about the catheter shaft, the stent disposed about the balloon to form an assembly, the assembly having an outer surface, the outer surface defining an outer diameter;
a sleeve, the sleeve disposed about the assembly, the sleeve comprising at least one layer of electroactive polymer material and at least one conductive member, the sleeve having an inner surface, the inner surface defining an inner diameter; and
a voltage source, the voltage source in electric communication with the at least one conductive member and the at least one layer of electroactive polymer material, the voltage source having an on state and an off state, in the on state the voltage source supplying a voltage to the sleeve.

2. The system of claim 1 wherein the sleeve has an expanded state and unexpanded state, in the expanded state the inner diameter is greater than the inner diameter in the unexpanded state, the sleeve being in the unexpanded state when the voltage source is in the off state, the sleeve being in the expanded state when the voltage source is in the on state.

3. The system of claim 2 wherein when the sleeve is in the unexpanded state the inner surface of the sleeve engages the outer surface of the assembly.

4. The system of claim 2 wherein the stent disposed about the balloon is in a crimped state or an uncrimped state.

5. The system of claim 3 wherein the assembly has an uncrimped state and a crimped state, in the uncrimped state the outer diameter being greater than the outer diameter in the crimped state, when the sleeve is in the expanded state the assembly is in the uncrimped state, when the sleeve is in the unexpanded state the assembly is in the crimped state.

6. The system of claim 1 wherein the sleeve further comprises a first electrical contact and a second electrical contact, the first electrical contact in electric communication with the electroactive polymer material, the second electrical contact in electrical communication with the conductive member.

7. The system of claim 1 wherein the sleeve further includes a lubricious coating applied to the inner surface of the sleeve.

8. The system of claim 1 wherein the sleeve has multiple layers.

9. The system of claim 8 wherein the multiple layers comprise at least one layer of non-EAP material.

10. The system of claim 1 wherein the sleeve has multiple conducting members.

11. The system of claim 10 wherein the multiple conducting members comprise wires.

12. The system of claim 11 wherein the multiple conducting members comprise ribbons.

13. The system of claim 11 wherein the multiple conducting members comprise both wires and ribbons.

14. A method of protecting an assembly comprising the steps of:
providing an assembly comprising a balloon catheter, the assembly having an outer diameter;
disposing a sleeve about the assembly, the sleeve comprising at least one layer of electroactive polymer material and at least one conductive member, the sleeve having an inner surface, the inner surface defining an inner diameter and having an expanded state and unexpanded state, in the expanded state the inner diameter is greater than the inner diameter in the unexpanded state;
setting a voltage source to an on state, the voltage source in electric communication with the at least one conductive member and the at least one layer of electroactive polymer material, the voltage source having an on state and an off state, in the on state the voltage source supplying a voltage to the sleeve whereupon the sleeve expands to an expanded state;
inserting the assembly into the region defined by the inner surface of the sleeve; and
setting the voltage source to an off state whereupon the sleeve contracts to an unexpanded state.

15. The method of claim 14 wherein the assembly further comprises a stent.

16. The method of claim 15 further including the step of applying a lubricious coating to the inner surface of the sleeve.

17. The method of claim 15 further including the step of applying a radially compressive force to the sleeve after the voltage source is set to an off state.

18. The method of claim 15 wherein the sleeve comprises multiple layers.

19. The method of claim 18 wherein the multiple layers comprise at least one layer of non-EAP material.

20. The method of claim 15 wherein the sleeve comprises multiple conducting members.
